(19) United States
(12) Patent Application Publication
(10) Pub. No.: US 2022/0194180 A1
(43) Pub. Date: Jun. 23, 2022

(54) DUAL DEVICE FOR ENVIRONMENTS DISINFECTION AND STERILIZATION OF AIR AND SURFACES

(71) Applicant: Juan Pablo Siri, Miami Beach, FL (US)

(72) Inventor: Juan Pablo Siri, Miami Beach, FL (US)

(21) Appl. No.: 17/558,886

(22) Filed: Dec. 22, 2021

Related U.S. Application Data
(60) Provisional application No. 63/199,374, filed on Dec. 22, 2020.

(51) Int. Cl. B60H 3/00; A61L 2/10; A61L 2/14; A61L 2/20; F24F 8/22; F24F 8/26

(52) U.S. Cl. CPC B60H 3/0078; A61L 2/10; A61L 2/14; A61L 2/202; A61L 2202/25; F24F 8/26; A61L 2202/11; A61L 2202/15; A61L 2202/14; F24F 8/22

(57) ABSTRACT

A dual device for environments disinfection and sterilization of air and surfaces comprising: an external casing with a mirrored inner surface; a top intake of polluted air; a bottom outlet of purified air, a centrifugal blower; at least one HEPA filter; at least one activated carbon filter; at least one UV lamp; an ionizer; an ozonizer and a buzzer.

### FIELD OF THE INVENTION

The present invention relates to devices and methods for purification, cleaning, disinfection and sterilization of air and surfaces in indoor environments and vehicles. In particular, the present invention relates to devices and methods that use multiple filtration and disinfection techniques and can be mounted on vehicles.

### STATE OF THE ART

It is known that numerous viruses, bacteria, fungi, spores, volatile organic compounds and particulate materials present in the air can cause bad odor and/or various health disorders such as bacterial infections, viral infections, allergies and other diseases. The air quality of the rooms where human beings perform their activities is of vital importance to maintain the safety and hygiene of such places.

On the other hand, the appearance of the new SARS-CoV-2 coronavirus, which mainly spreads by airborne transmission by aerosols in the air, has renewed interest in the development of air and surface disinfection devices that can also be used in vehicles, especially public transport vehicles such as buses, trains, ships, etc.

In this sense, numerous technologies, methods and strategies have been developed to eliminate air pollutants and disinfect rooms, however, all of them present some disadvantages.

Among the physical methods the use of HEPA filters can be mentioned. These filters consist of a physical barrier that is capable of stopping and retaining particles by different mechanisms. Other kinds of filters, such as activated carbon filters, are capable of absorbing certain molecules such as volatile organic compounds on their surfaces. Another widely used physical method is based on the use of ionizing radiation (such as UV light) for the elimination of microorganisms, which acts by removing the binding electrons of macromolecules that are irreplaceable for the life of cells, becoming unviable if the irradiated dose is high enough. In addition, through the use of air ionizers, it is possible to neutralize the electrostatic charges that keep the fine particles in suspension and therefore achieve their elimination.

On the other hand, among the chemical methods, the use of ozone is among the most relevant ones. This chemical compound is a strong oxidant. Its main advantage lies in the conversion of ozone into oxygen, without leaving any harmful chemical residue. More recently, the use of photocatalytic systems has been widely spread. UV light is used to promote the formation of highly reactive and short-lived chemical species on their surface, which are capable of eliminating contaminants.

However, the use of these systems separately is usually not effective to achieve the goal of complete removal pollutants from the environment.

Among the main disadvantages of these systems are the low odor absorption capacity of HEPA filters and the possible proliferation of the microorganisms retained in the filters under the right conditions. UV lamps installed in air conditioning equipment usually do not achieve an adequate level of disinfection due to the high speed of the air flow that passes through the purification devices. Ozone, despite being an excellent disinfectant, is harmful in high concentrations and can irritate the upper respiratory tract in concentrations greater than 0.1 ppm.

In the state of the art, some documents that describe methods and devices for disinfection of air wherein various technologies are combined can be found. For example, the document CN105737262 B discloses an air purifier comprising an in-line intelligent induction and control device and guiding device, activated carbon filters, HEPA filters, an activated carbon photocatalyst, negative ion generator and ozone generator. In addition, the device autonomously detects the indoor air quality through various sensors and automatically adjusts the purification parameters of the equipment according to the air quality in automatic mode. However, it has an important disadvantage, being designed to be located on the floor of a room, its IR sensor may not detect the presence of living beings in their sterilization mode and seriously affect their health. On the other hand, it presents a major design problem; since ozone is denser than air, it has a short half-life and the device takes the air from a zone near the floor and drives it to a low height, ozone cannot reach greater heights and only what is below the outlet of the device is sterilized.

Document WO2018/106809 A3, discloses a device that measures air quality parameters at the inlet and the outlet of the device and, based on the data collected, executes actions. In the detailed description of the invention, it is disclosed that said device may comprise technical characteristics similar to those previously mentioned. However, the document does not disclose how to order the various cleaning and disinfection methods mentioned to maximize their efficiency.

The utility model CN203785115 U provides a negative ion air purifier, said device comprises a fan, a negative ion generator, HEPA filters, activated carbon fiber filters, a multiplicity of UV lamps, and an antibacterial filter with silver nanoparticles. On the other hand, utility model CN205615319 U discloses a purifying device to be used in vehicles, comprising nano silver ion filters, activated carbon composite filter screen, HEPA filter screen and anion generator. Nonetheless, these devices do not include an ozone generator so they are not capable of generating adequate disinfection of surfaces.

In addition, state-of-the-art devices that purify the air from the ground present the impossibility of aspiring the polluted air in height and returning it purified to the level of the respiratory tract. During the day, human beings generate aerosols at greater heights than 1.6 meters, and they breathe the polluted air at the same height.

As can be seen, there is still a need for an air and surface purification device and method, installable in indoor enviroments and vehicles, that combines different decontamination strategies in such a way as to ensure maximum disinfection and air quality.

The present invention solves the problems identified in the state of the art, providing a device that comprises an external casing with mirrored inner surfaces; a top intake of polluted air; a bottom outlet of purified air; a centrifugal blower; at least one HEPA filter; at least one activated carbon filter; at least one UV lamp; an ionizer; an ozonizer and a buzzer which, preferably located higher than 2 m, works in air purifying mode, drastically lowering the pollutant load or in sterilization mode, generating ozone until the elimination of organic compounds, viruses and bacteria.

The present invention provides a device that, operating in its sterilization mode, forcefully and effectively prevents the ozone generation in presence of a living being in the room.

None of the documents of the state of the art provide application examples that demonstrate their effectiveness in the purification of rooms; they do not have casings or covers with mirrored or reflective surfaces to maximize the sterilizing effect of UV light; nor do they reveal the ideal distance from the ground for the installation of said devices, a factor that is key to air disinfection.

### BRIEF DESCRIPTION OF THE FIGURES

FIG. 1 shows the differences between the commercially available turbine and the modified one.

FIG. 2 shows a cross-sectional diagram of the dual device for air disinfection and sterilization of environments and surfaces.

FIG. 3 shows the removal efficiency of S. aureus.

FIG. 4 shows the removal efficiency of A. niger.

### BRIEF DESCRIPTION OF THE INVENTION

A dual device for environments disinfection and sterilization of air and surfaces, the main object of the present invention, comprises: an external casing with mirrored inner surfaces, preferably polished metal, more preferably polished stainless steel; a top intake of polluted air; a bottom outlet of purified air; a centrifugal blower; at least one HEPA filter, preferably H11, H13 and/or H14 grade; at least one activated carbon filter, preferably granular, fiber or powdered activated carbon; at least one UV lamp; an ionizer, preferably plasma; an ozonizer; a buzzer, which preferably generates a light signal and a buzz that makes it impossible to remain in the room to be sterilized.

In a preferred embodiment, it also comprises a photocatalytic air filter. More preferably it comprises a mesh structure coated by TiO₂ which is irradiated by UV lamps.

Preferably, said dual device for environments disinfection and sterilization of air and surfaces, also comprises a remote-controlled automatic control system and a presence sensor, preferably an infrared detector, which detects the absence of living beings in the room where it is installed, enabling the operation of said ozonizer to comply with the sterilization mode of operation. Said sterilization mode also activates said buzzer that generates a light signal and a buzz that makes it impossible to remain in the room to be sterilized. Even more preferably, the device comprises two operation modes: standard and sterilization; the ozonizer only works in sterilization mode.

Said UV lamps are selected from the group comprised by: UVA/UVC lamps and FAR UV-C lamps. In turn, these lamps are also selected from the group that includes: LED technology lamps and mercury vapor lamps.

In a preferred embodiment, said blower or mechanism to displace air comprises two centrifugal turbines commanded by a single electric motor located between said turbines that operates at 2800 rpm, generating an air flow of at least 2000 m³/h. Preferably, said centrifugal turbines have a design with reduced air intake ducts.

Preferably, the device operates at a height of at least 2 meters attached to a wall, aspiring the air through its upper section and driving the purified air downwards, in such a way that in its sterilization mode the ozone reaches all the furniture surfaces and objects that are below two meters in height, achieving the sterilization of the surfaces.

In another preferred embodiment, said UV lamps, said activated carbon filter, said ionizer and said ozonizer are installed on aluminum profiles inside said casing which is constituted by the air conditioning ducts of a vehicle, where said blower is the impeller of said air conditioning system. In addition, said activated carbon filter, said HEPA filter and said photocatalytic filter are manufactured to fit into said ducts so that the circulating air passes through them. Preferably, a touch screen, the presence sensor, and all the electronic components that are needed for operation, are installed according to the requirements and possibilities in each vehicle.

Another object of the present invention is a method of purification, cleaning and disinfection of air and surfaces for indoors that uses the device of the present invention that comprises the following steps: aspiring the air to purify from the top; forcing the air aspired in the previous step to pass through the activated carbon filter, through the HEPA filter, through a UV lamp, through the ionizer and expelling the purified and disinfected air through a lower outlet. Preferably, the device is installed at a distance of at least 2 m and less than 5 m from the ground or floor.

More preferably, said device is installed at a height between 2 and 2.5 m from the ground; even more preferably, said device is installed at a height of 2.3 m.

In turn, the method preferably comprises the automatic activation of said ozonizer when its remote-controlled automatic control system detects the absence of living beings in the room.

When the ozonizer is operating, producing ozone, this process becomes an air and surface sterilization process. In this case, the device of the invention is said to be in a sterilization mode of operation.

### DETAILED DESCRIPTION OF THE INVENTION

The dual device for environments disinfection and sterilization of air and surfaces of the present invention is characterized by combining various technologies for cleaning and sterilizing air and surfaces in an orderly and efficient manner in order to have excellent air and surfaces disinfection performance.

The device combines physical and chemical methods for the retention and removal of particulate matter and microorganisms from the air and sterilization of surfaces.

Preferably, the device uses a combination of activated carbon filters (101), HEPA filters (102), photocatalytic filters (103), UV lamps (104), ionizer (105) and ozonizer (106).

Additionally, the device of the present invention comprises a touch screen, which allows controlling the operation of the equipment and allows monitoring the operation of the equipment, temperature, program and air condition; remote control, to perform all basic operations from a distance; presence sensor, which allows the ozonizer to work in the absence of living beings; a blower or mechanism to displace air (107) and supports for its installation on a wall. The device of the present invention further comprises an external casing (108) that contains all the pollutant retention and elimination systems, the mechanism to displace air and all the electronics necessary for its operation. Said casing, comprises mirrored inner surfaces and is designed in such a way that it is capable of reflecting the ultraviolet light emitted by the UV lamps, maximizing their efficiency while avoiding direct contact of radiation with humans.

The device works in such a way that ambient air is aspired into the casing (108) through a top intake of polluted air (109) located at upper section of the device, thanks to the pressure gradient generated by the mechanism to displace air (107). Then, the air passes through the activated carbon filters (101), the HEPA filters (102), and the photocatalytic filters (103). Once inside the device, the air is irradiated with ultraviolet light. In turn, the ultraviolet lamps (104) irradiate the HEPA filter (102) to prevent the proliferation of fungi and bacteria in it, while also irradiating the structure that supports the photocatalyst to produce the desired photoreactions. Inside the casing (108), negative ions are introduced into the air by the ionizer (105) and ozone is introduced by the action of the ozonizer (106). Finally, the filtered, disinfected and loaded with anions and ozone air, interacts with the mechanism to displace air (107), which forces it out of the casing through a bottom outlet of purified air (110) located at the lower section of the device.

In another embodiment, the device of the present invention can be installed in all types of vehicles that have an air conditioning system such as buses, trains, cars, boats, etc.

In this way, said casing is constituted by the walls of the air conditioning ducts, while the mechanism to displace air is the same blower or impeller used by the preexisting air conditioning system of the vehicle. According to this embodiment, the UV lamps (104), the ionizer (105) and the ozonizer (106) are installed on aluminum profiles that are located inside the air conditioning ducts; while the activated carbon (101), HEPA (102) and photocatalytic (103) filters are manufactured to fit inside of said ducts so that the circulating air passes through them. The location of the touch screen, presence sensor, and electronic components necessary for its operation are decided based on the needs and possibilities of each vehicle.

In this document, disinfection is understood as any action executed to reduce the load of viruses, bacteria, fungi, microorganisms, organic gases and particles. It is understood from a general perspective, also including the concept of purification.

Sterilization is understood herein as the removal of microorganisms or viruses. It should be clarified that the action of the device of the present invention eliminates microorganisms and viruses from a room and its exposed surfaces when the ozonizer is activated, in its sterilization mode of operation, as long as it is allowed to operate long enough.

### Activated Carbon Filters

Activated carbon is a highly porous material that, through the phenomenon of surface adsorption, retains gases and vapors from the air, for example, volatile organic compounds, radon, and NO₂, among others.

In a preferred embodiment of the present invention, the activated carbon filters (101) comprise granular activated carbon housed in a honeycomb-like cell structure, where the volume of said cells is partially filled (20% of the volume of the cell) with activated carbon granules. In this way, a low-pressure drop and adequate distribution of the granules is achieved as a result of a homogeneous thickness of adsorbent material. The formation of plugs or preferential channels for the passage of gases is also avoided.

In another embodiment, the activated carbon filter (101) comprises the use of activated carbon fibers. This morphology has unique characteristics compared to granular or powdered activated carbon. The thin fibrous shape ensures faster absorption and is very easy to handle.

### HEPA Filters

HEPA (High-Efficiency Particulate Arresting) filters are composed of a mat of randomly arranged fibers. The fibers are typically composed of fiberglass, cotton or polyester, with diameters between 0.5 and 2.0 μm. HEPA filters are designed to effectively stop very fine particles, but they do not retain gases or odor molecules.

In the device of the present invention, the HEPA filters (102) are irradiated by an ultraviolet lamp that produces light in a wavelength range of 254-280 nm. This germicidal process generates the total inactivation of the viruses and bacteria that are trapped in the fibers of the HEPA filter (102). If this inactivation is not generated, mold, yeasts and bacteria generate colonies in the filters and over time they can return to the environment. The device of the present invention comprises HEPA filters (102) wherein said HEPA filters grades are selected from the group comprised by H11, H13 and H14. In a preferred embodiment, it uses HEPA H14 filters.

### Photocatalytic Filter

The photocatalytic filtering process comprises an air filter that requires to be illuminated by UV light, the filter (103) is irradiated by the lamps, generating photocatalysis.

It is known that UV radiation over titania induces the formation of electron-hole pairs, whose charge carriers react with chemical species such as H₂O, OH⁻ and O₂ to produce hydroxyl radicals (OH.), superoxide radical anions (O2.−), and H₂O₂ that contribute to the decomposition of organic compounds on the surface of TiO₂.

The photocatalytic filter (103) comprises a support and a photocatalytic material. In a preferred embodiment of the invention, said support comprises metal mesh with large opening size that supports TiO₂ powder. Said metal mesh has a very open weave that allows UV rays to pass without generating interference and thus allows ultraviolet radiation to also irradiate the surface of the HEPA filters (102). Two processes are generated at the same time, the photocatalysis and the inactivation of microorganisms in the HEPA filter (102).

### Ultraviolet Light

Ionizing electromagnetic radiation in adequate doses can generate cellular damage by direct action (damage to important macromolecules such as DNA, RNA, enzymes) or mechanisms that involve indirect action (damage to the cell membrane, formation of free radicals by homolytic breakdown of the water present in the cytoplasm, etc). When the radiation dose is high enough, the damage to the cells is too great and they are unable to repair themselves, becoming unviable.

On the other hand, ultraviolet radiation is capable of irradiating HEPA filters at the same time as the photocatalyst filter, where the chemical species that contribute to the decomposition of organic matter on its surface are generated.

The UV lamps (104) are located in such a way that, in their position, the radiation received by the HEPA filter (102) exceeds 50 W/cm² and in turn irradiates the turbine rotor of the mechanism to displace air (107) eliminating any microorganism that adheres to it, thanks to the interior mirrored surface of the casing (104).

Said UV lamps (104) comprise LED technology lamps or mercury vapor lamps. In one embodiment, said UV lamps (104) comprise UVC-FAR type lamps. In a preferred embodiment, said UV lamps (104) are of the UVA/UVC type with LED technology.

### Anions or Negative Ions

Dust particles, pollen, bacteria and other allergens have a positive surface charge and are capable of remaining in suspension in the environment thanks to electrostatic interactions. Negative ions act by neutralizing this charge and sometimes causing their agglomeration, promoting their precipitation due to gravity.

The ionizer (106) comprises a plasma anion generator. In addition to eliminating 99% of all suspended particles of less than 2.5 microns, plasma can neutralize viruses and bacteria by destroying their molecular structure.

### Ozone

Ozone (O₃) is a well-known disinfecting agent which has been widely used in various applications because it is a highly oxidizing compound that destroys organic substances, bacteria molecules, molds, etc.; therefore, it is capable of sterilizing the air and eliminating toxic gases and odors. Ozone owes its great oxidizing capacity to the fact that its third oxygen atom is a loose radical that reacts with volatile organic compounds, neutralizing odors and certain gases, producing oxygen (O₂) as the main by-product. It has germicidal properties and is capable of destroying all kinds of toxins, bacteria, fungi and viruses present in an environment. Being a gas, it is capable of facilitating disinfection tasks in upholstery and fabrics as well as in areas of difficult access.

In one embodiment of the invention, the ozonizer (106) comprises corona discharge ozone generators. In another embodiment of the invention, the ozonizer (106) comprises UVC-O3 lamps specifically designed to maximize ozone generation.

The density of ozone is 2.1 kg/m³ while that of air is only 1.2 kg/m³. For this reason, many of the devices that constitute the state of the art do not work, especially if they are located on the floor.

The device of the present invention is designed to operate at a height of more than two meters and has directing fins to direct the purified air that comes out from under said casing in various directions and at angles close to horizontal to achieve its optimal distribution. In this way the ozone falls on the surfaces of furniture and objects, sterilizing them.

### Buzzer

The device of the present invention has a buzzer that, when the sterilization mode of operation starts, turns on the flashing red light and generates at least 80 dB of sound and a light that can be red and flash-led type.

The device of the invention also has a mechanism to displace air (107), or blower, which comprises an electric motor that is attached to moving surfaces. Said mobile surfaces are set in motion by said motor, displacing the air necessary for the correct operation of the device of the present invention. In a preferred embodiment of the present invention, said mechanism to displace air (107) comprises an electric motor with two turbines. In an even more preferred embodiment of the present invention, the device comprises a blower (similar to those used in air conditioning equipment) composed of two centrifugal turbines controlled by a single electric motor located between said turbines that operate at 2800 rpm. According to this embodiment, the turbines comprise an inner casing and a multiplicity of blades.

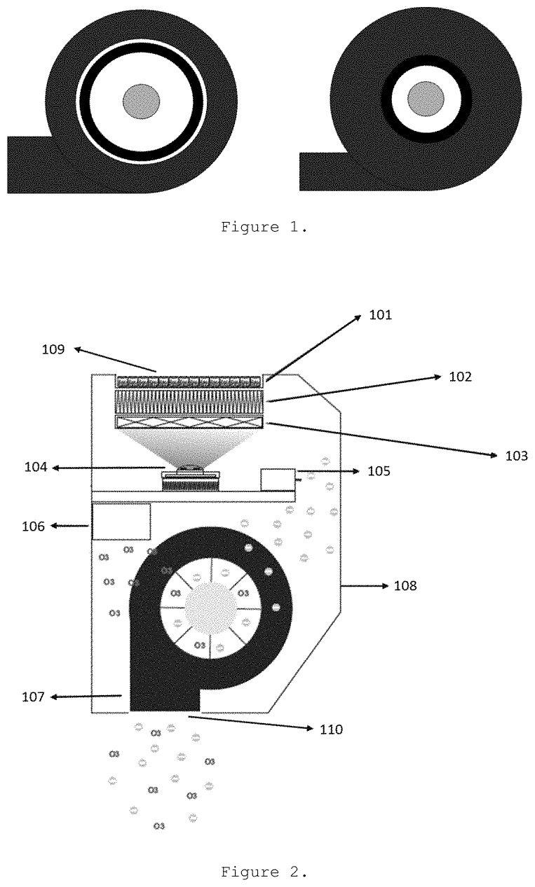

In an even more preferred embodiment of the invention, the mechanism for displacing air (107) is based on a commercially available device (FIG. 1, diagram on the left), in which its design was modified by reducing the air intake ducts of air and preventing the air blown by the turbine from colliding with surfaces that may generate sound due to the effect of the high-pressure air flow against a rigid surface, as shown in the diagram on the right in FIG. 1. In this way the equipment is capable of producing 400% higher air flow with the engine running at 2800 rpm, maintaining the same noise level.

In one embodiment, the device of the invention comprises at least two modes of operation: standard and sterilization. In standard mode, the UV lamps (104) and the ionizer (105) operate, while air is circulated through the activated carbon (101), HEPA (102) and photocatalytic (103) filters. In the sterilization mode, the ozonizer (106) is also turned on. The sterilization mode is activated by the user when leaving the room or vehicle. A timer can be programmed so that the operation of the ozonizer is interrupted before people return to the room or vehicle. The buzzer turns on at the same time as the ozonizer. The presence sensor works as a safety device, immediately interrupting the operation of the ozonizer if the presence of people or pets is detected.

Another object of the present invention is a method of purification, cleaning and disinfection of air and surfaces for indoors that uses the device of the present invention, characterized in that said device is preferably installed at a minimum distance of 2 m from the floor and comprises the following steps:
a. aspiring the air from the room to purify through a top intake;
b. forcing the air aspired in step “a” to pass through the activated carbon filter, through the HEPA filter, through a UV lamp, through the ionizer and
c. expelling the purified and disinfected air through a bottom outlet.

Furthermore, said method is characterized in that it is a method of sterilizing the air in the room where it is installed, which comprises the automatic operation of said ozonizer when its remote-controlled automatic control system detects the absence of living beings in the room.

The device of the invention is preferably installed at a height of between 2 and 2.5 m from the ground, always preferably less than 5 m. In an even more preferred embodiment of the invention, the device of the invention is installed at a minimum distance of 2.3 m from the ground.

### EXAMPLES

### Example 1

An Application Example of the Present Invention is Described in Detail Below without Intending to Limit the Claims. The Device Described in this Example Allows a Person Skilled in the Art to Reproduce it.

### Construction of a Dual Device for Environments Disinfection and Sterilization of Air and Surfaces of the Present Invention.

The device of the present invention was built from a stainless-steel support, in which, a UV LED lamp (104), an ozonizer (106) and a plasma ionizer (105) were fixed by specially designed screws and sockets. Said support is attached to a main body by welding, allowing the device to be attached to a wall by using bolts. A stainless-steel casing (108) polished on its inner surfaces closes the device and is attached by pressure fittings and security screws to said support. On the horizontal face of the casing (108), a CPU with a touch screen was attached to the outside of the device, which allows controlling the operation of the equipment and allows processing the information from the temperature, program selected and air condition sensors. In addition, this CPU is controlled remotely or by touch, to execute all the basic operations remotely. A buzzer was mounted, connected to said CPU, which when the sterilization operating mode starts, turns on the flashing red light and generates a sound of 80 dB and turns on a red flash-led light. Said device also comprises a remote-controlled automatic control system and a presence sensor, which detects the absence of living beings in the room where it is installed, enabling the operation of said ozonizer (106) to fulfill the sterilization function.

Said presence sensor is an infrared motion sensor with a range of 4 meters and a 180-degree opening, which detects the absence of living beings in the room where it is installed, enabling the operation of said ozonizer (106) to comply with the sterilization mode of operation, where said sterilization mode also activates said buzzer that generates a light signal and a buzz that makes it impossible to stay in the room to be sterilized.

As shown in FIG. 2, said casing (108) contains all the contaminant retention and elimination systems (101, 102, 103, 104, 105 and 106), the mechanism to displace air (107) and all the necessary electronics for its operation. Inside, it comprises mirrored stainless steel inner surfaces and is designed in such a way that it is capable of reflecting the ultraviolet light emitted by the lamps, maximizing their efficiency.

In particular, said activated carbon filter (101) comprises granular activated carbon housed in a honeycomb-like cell structure, where the volume of said cells is partially filled (20% of the volume of the cell) with activated carbon granules. This avoids the reduction of the air flow and inactivates harmful gases. In addition, said filter is located next to the top intake of polluted air (109) of the casing (108), as shown in FIG. 2. The HEPA filter (102) comprises H14 type filter panels that are capable of retaining 99.995% of the particles and microorganisms from the air that passes through it. The photocatalytic filter (103) comprises a support, and a photocatalytic material. In particular, said support comprises a metal mesh with large opening size that supports TiO₂ powder, which is impregnated on the mesh. Said metal mesh has a very open weave that allows UV rays to pass through without generating interference and thus allows ultraviolet radiation to reach the HEPA filters (102).

In addition, said UV lamp (104) is a UVA/UVC type LED lamp and is located in such a way that in its position the radiation received by the HEPA filter (102) exceeds 50 W/cm², and that it also irradiates the photocatalytic filter (103) managing to activate the photocalytic process. The mechanism to displace air (107) is a motor that drives two centrifugal blowers and that rotates at 2800 rpm. Regarding the ionizer (105), it is a negative plasma ion generator, while the ozonizer (106) is a corona discharge ozone generator.

Regarding the mechanism to displace air (107), it comprises an electric motor that is bolted. This motor rotates two shafts driving separate centrifugal blowers. Said blowers are two centrifugal turbines controlled by a single electric motor which is located between said turbines. Said turbines comprise an inner casing and a multiplicity of blades. Said mechanism (107) is located close to the air outlet (110) of the casing (108).

Starting from a commercially available mechanism, its design was modified by reducing the air intake ducts of said commercially available device. In this way, the air blowing from the turbine does not collide with surfaces that could generate sound produced by the effect of the high-pressure air flow against a rigid surface. In this way, the equipment is capable of producing an air flow 400% higher, maintaining the same noise level. The air flow that is achieved with these turbines exceeds 2000 m³/h.

The device comprises two operation modes: standard mode and sterilization mode. In the standard mode the UV lamps (104) and ionizer (105) operate and air is circulated through the activated carbon (101), HEPA (102) and photocatalytic (103) filters. In the sterilization mode, the ozonizer (106) is also turned on. The sterilization mode is activated by the user when leaving the room or vehicle. In addition, it has a timer, which can be programmed in such a way that the operation of the ozonizer (106) is interrupted before people return to the room or vehicle. The presence sensor works as a safety device, immediately interrupting the operation of the ozonizer (106) if the presence of people or pets is detected.

### Example 2

Analysis of the Microbiological Quality of the Air Before and after the Installation and Operation of the Dual Device for Environments Disinfection and Sterilization of Air and Surfaces in a Controlled Space.

### Specific Test

Systemic evaluation of the microbiological quality of the air in indoor environments to determine the performance of the device of the present invention in the microbiological decontamination of the air.

### Methodology

Molecular detection of bacteria using the 16S marker and molecular detection of SARS-CoV-2 from the genetic material extracted from the samples.

### Experimental Design

The device of the Example 1 was located in a 0.7 m³ cabin conditioned to be able to execute the tests under controlled conditions (without air currents and isolated from the movement of people). By means of a spray bottle, a suspension of a microbial community of known composition and a suspension of synthetic viral particles of SARS-CoV-2 virus were added to the environment.

Four samples were taken from the cabin: T0 (before adding the inoculums, control of the basal level of contamination), T1 (after adding the inoculums in the absence of the equipment, positive control with maximum contamination), T2 (after adding the inoculums in the presence of the device after 30 min of operation) and T3 (after adding the inocula in the presence of the device after 60 min of operation).

The samples taken were processed individually to purify the genetic material from the microorganisms and then detect them by the molecular techniques mentioned in the methodology.

### Results

The obtained results are summarized in the following table:

TABLE I

| | | | | | Bacteria |
|---|---|---|---|---|---|
| | | | Operation | SARS-CoV-2 | molecular |
| | | | time | molecular | detection |
| Sample | Inoculum | Device | (min) | detection | by 16S |
| T0 | Non | Absent | NA | Not | Not |
| | Applied | | | detectable | detectable |
| T1 | Applied | Absent | NA | Detectable | Detectable |
| T2 | Applied | operating | 30 | Detectable | Detectable |
| T3 | Applied | operating | 90 | Not | Not |
| | | | | detectable | detectable |

*NA: Not applicable.

### Determination of Bacterial Load in the Air by 16 S:

The 16S molecular detection of the microbial genomes was able to amplify the DNA extracted from the artificially inoculated suspension in the 0.7 m³ cabin in the absence of the device (sample T1).

The result reveals that after operating the device, for 30 and 90 minutes in a controlled space of 0.7 m³, it reduces by 99.7% (sample T2, 30 min) and at NON-DETECTABLE levels (sample T3, 90 min), the microbial DNA present in the environment.

### Determination of SARS-CoV-2 Load in the Air:

The molecular detection of the SARS CoV-2 virus was able to amplify the RNA extracted from the suspension of synthetic viral particles artificially inoculated in the 0.7 m³ cabin in the absence of the device (sample T1).

The result of the analysis reveals that after operating the device, for 30 and 90 minutes in a controlled space of 0.7 m3, it reduces by 99.9% (sample T2, 30 min) and at NON-DETECTABLE levels (sample T3, 90 min), the SARS-CoV-2 genome present in the environment.

### Conclusions

The obtained results indicate that the device operating in a controlled environment of 0.7 m³, not exposed to fluctuations in air currents and artificially loaded with a certain amount of bacteria and synthetic viral particles of the SARS-CoV-2 virus, is capable of reducing, by 99% (after 30 min of operation) and at non-detectable levels (after 90 min of operation), the applied microbiological load.

### Example 3

Analysis of Removal of Microorganisms from the Air without Ozonation.

### Trial Objectives

The Objective of the Trial is to Evaluate the Effectiveness of the dual device for environments disinfection and sterilization of air and surfaces in the removal of microorganisms present in the air.

### Methodology

The microbiological air quality of a model room of approximately 300 m³ was evaluated after closing the room and operating the dual device for environments disinfection and sterilization of air and surfaces for 24 continuous hours, using a standardized qualitative test suggested by the Pharmacopoeia of United States (Chapter <1116>).

To evaluate the microbiological quality of the air, open Petri dishes containing a solid culture medium (TSA) were exposed for 4 hours. The Petri dishes were exposed in 4 different points of the room. The selected sites were: behind the air intake of the equipment (site 1), in front of the outlet at a short distance (site 2), in front of the outlet at a greater distance (site 3) and in a corner away from the equipment (site 4). In this way, it was possible to evaluate possible effects of microorganism concentration gradients as a function of the placement distance and operation of the equipment. A dish was placed in a sterile environment (laminar flow cabinet) as a negative control. Subsequently, the dishes were incubated in a culture oven for 72 hours at two different temperatures, 25° C. and 32° C., to favor the growth of fungi and bacteria, respectively. The number of colonies grown on the dishes before and after 24 hours of continuous operation of the equipment was compared.

Table II: Colonies present on the dishes after 72 hours of incubation at adequate temperatures before turning on the device (0 hours) and after closing the room and operating the device for 24 hours.

TABLE II

| Incubation temperature | |
|---|---|
| 25° C. | 32° C. |

| Time | 0 h | 24 h | 0 h | 24 hs |
|---|---|---|---|---|
| Site 1 | 39 colonies | 5 colonies | 10 colonies | 3 colonies |
| Site 2 | 50 colonies | 7 colonies | 39 colonies | 6 colonies |
| Site 3 | 38 colonies | 7 colonies | 45 colonies | 3 colonies |
| Site 4 | 55 colonies | 4 colonies | 45 colonies | 2 colonies |

### Results

It was observed that, regardless of the culture temperature, the presence of microorganisms significantly decreased after 24 hours of operating the equipment. In turn, no significant changes were observed in the concentration of microorganisms depending on the placement distance of the dishes.

Despite the qualitative nature of the test, a count of the microorganisms colonies that grew on the dishes was made, in order to obtain an estimated result of the removal percentage of microorganisms achieved by the equipment after 24 hours of operation. Table II shows the colony results per dish obtained for the 4 sampling sites, using both incubation temperatures. It can be observed that regardless of the sampling site and incubation temperature, the dual device for environments disinfection and sterilization of air and surfaces removed approximately 80% of the microorganisms present in the air in the closed model room.

### Conclusions

The device evaluated showed significant removal of the number of microorganisms present in the air after at least 24 hours of continuous use. Removal of airborne microorganisms is estimated to be at least 80% of the environmental microorganisms originally present in the room.

### Example 4

Analysis of Microorganisms Removal from the Air and Surfaces with Ozonation. Device from Example 1 Located on a Wall of the Room at 2.3 m High.

### Trial Objectives

The objective of the trial is to evaluate the effectiveness of the dual device for environments disinfection and sterilization of air and surfaces in the removal of microorganisms present in the air and surfaces of a closed room.

The microbiological air quality and surfaces of a model room of approximately 300 m³ was evaluated after closing the room and operating the dual device for environments disinfection and sterilization of air and surfaces, using standardized qualitative tests suggested by the Pharmacopoeia of United States (Chapter <1116>). The equipment was installed in the room. The quality of the air and various surfaces was evaluated after 24 hours of continuous operation of the equipment, including a 2-hour ozonation cycle. The obtained results were compared with the microbiological quality of air and surfaces of the same room before operating the equipment.

To evaluate the microbiological quality of the air, open Petri dishes containing a solid culture medium (TSA) were exposed for 4 hours. The Petri dishes were exposed in 4 different points of the room. The selected sites were: below the air outlet of the equipment (site 1), in front of the device at short distance (site 2), in front of the device at a greater distance (site 3) and in a corner away from the equipment (site 4). In this way, it was possible to evaluate possible effects of microorganism concentration gradients as a function of the placement distance and operation of the equipment. A dish was placed in a sterile environment (laminar flow cabinet) as a negative control. Subsequently, the dishes were incubated in a culture oven for 72 hours at 25° C. The number of colonies grown on the dishes before and after 24 hours of continuous operation of the equipment was compared.

To evaluate the microbiological quality of the surfaces, swabs were made in the same 4 sites of the room. Swabs were performed in pre-defined and labeled 10 cm×10 cm sections. Afterwards, the bacteria were extracted from the swabs by immersing them in 5 ml of phosphate buffer and vortexing. 200 μL of the suspension of bacteria recovered from the surfaces were taken and inoculated in Petri dishes containing solid culture medium (TSA). The dishes were incubated in a culture oven for 72 hours at 25° C.

### Results

Removal of Microorganisms from the Air

It was observed that the presence of microorganisms decreases significantly in the air after 24 hours of operating the equipment. In turn, significant changes were observed in the concentration of microorganisms depending on the placement distance and operation of the equipment.

The dishes placed closer to the equipment showed higher percentage of microorganisms removal than the dishes placed in remote regions. It should be noted that the equipment was programmed so that the motor that performs air recirculation was in low power mode.

Despite the qualitative nature of the test, a count of the microorganisms colonies that grew on the dishes was made, in order to obtain an estimated result of the removal percentage of microorganisms achieved by the equipment after 24 hours of operation. Table III shows the colony results per dish obtained for the 4 sampling sites. It can be observed that the dual device for environments disinfection and sterilization of air and surfaces removed a maximum of 80% of the microorganisms present in the air in the closed model room, in regions close to the air outlet of the device. However, it should be noted that this is an estimate, since the number of bacteria present in the air could be higher, since no type of air capture system was used and the microorganisms that grew on the dishes are the product of static deposition.

Table III: Colonies present on the dishes after 72 hours of incubation at 25° C. before turning on the device (0 hours) and after closing the room and operating the device for 24 hours.

TABLE III

| | Incubation temperature | | |
|---|---|---|---|
| | 25° C. | | |
| Time | 0 h | 24 h | % removal |
| Site 1 | 60 colonies | 9 colonies | 85.0% |
| Site 2 | 26 colonies | 8 colonies | 70.0% |
| Site 3 | 24 colonies | 9 colonies | 62.5% |
| Site 4 | 20 colonies | 12 colonies | 40.0% |

### Surface Microorganism Removal

The presence of microorganisms on the surfaces decreases significantly after continuous use of the device, even reaching a complete removal of microorganisms in some of the sampling sites. It should be noted that after the 2-hour ozonation cycle, the aroma of ozone was perceived in the environment.

### Conclusions

An evaluation of the equipment efficiency in the disinfection of air and surfaces was performed in a closed room of approximately 300 m³. The evaluated device presented a significant removal of the microorganisms present in air and surfaces after at least 24 hours of continuous use. The evaluated device produced a significant reduction of the quantity of microorganisms present in air and surfaces after at least 24 hours of continuous operation. It is estimated that the microorganisms removal from the air is approximately 80% of the microorganisms originally present in the room environment, while on surfaces the removal was even higher, reaching values close to 100%.

### Example 5

Analysis of Microorganisms Removal from the Air and Surfaces with Ozonation. Device from Example 1 Located on a Wall of the Room at 0.5 m and 1 m High.

The tests of Example 4 were repeated placing the device of the invention at a height of 0.5 m and on a table at a height of 1 m. The results were poor, the device barely achieved a 30% removal or less. Under these conditions, the device was not able to sterilize surfaces.

### Example 6

Microbial removal efficiency achieved by the device of Example 1

Test challenge organisms: Bacteria: Staphylococcus aureus; Fungi: Aspergillus niger.

TEST: Microbial Removal Efficiency

Contracted Laboratory: Environmental Diagnostic Laboratory 4911 Creekside DR., Suite C—Clearwater, Fla. 33760

Methodology: to determine the microbial removal efficiency of the device of the present invention, a known concentration of the selected challenge organisms Staphylococcus aureus and Aspergillus niger were aerosolized through the device. Air samples were collected both when the device was switched off (device not operating) and switched-on (all components of the device were operating) utilizing the settling plate method at two different time intervals (0 and 60 minutes) with Typic Soy Agar (TSA) and Malt Extract Agar (MEA) microbial culture media for S. aureus and A. niger, respectively. Samples for pretest sterility verification of the test chamber were collected in addition to the negative and positive control samples. The viability and concentration of the suspension utilized for the aerosolization of the test organisms (S. aureus and A niger) was determined by serial dilution technique.

### Controls:

### Negative Controls:

Reagent Water Lot:476425 Exp:06-26-2021

### Positive Controls:

Staphylococcus aureus ATTC: 25923 Exp:06-30-2021

Aspergillus niger ATTC:6275 Exp:01-31-2022

### Microbiological Media Used:

TSA: Lot #: 135372 Exp:06-07-2021

MEA: Lot #:47409 Exp:05-17-2021

### Challenge Concentration:

Staphylococcus aureus 50.8*10⁴

Aspergillus niger 25.0*10²

### Observations:

All the experiments were conducted under standard laboratory conditions. Table IV records the sterility verification of the environmental test chamber. Table V contains the results of the efficacy of the tested device.

### Table IV: Sterility Verification

TABLE IV

| Trial | Bacteria | Fungi |
|---|---|---|
| Sterility | BDL | BDL |
| verification | | |

*BDL: Below detection limit

### Table V: Microbiological Efficacy Results

TABLE V

| | |
|---|---|
| Pre-treatment | Post-treatment |

| | Colonies | Colonies | Colonies | Colonies |
|---|---|---|---|---|
| Challenge | (CFU/m²) | (CFU/m²) | (CFU/m²) | (CFU/m²) |
| organisms | 0 min | 60 min | 0 min | 60 min |
| S. aureus | 899422 | 373 | 2986 | BDL |
| A. niger | 8584 | 1866 | 187 | BDL |

*BDL: Below detection limit

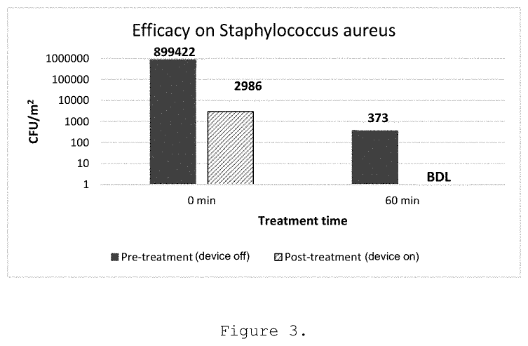

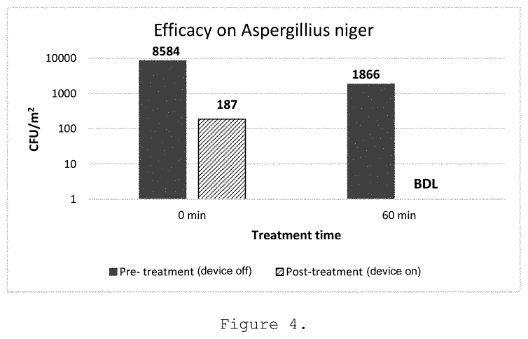

FIGS. 3 and 4 show efficacies in microorganisms removal: S. Aureus in FIG. 3 and A. Niger in FIG. 4.

### Conclusion

This device is classified as effective against microbes (Staphylococcus aureus and Aspergillus niger). This test proves the sterilizing ability of the equipment.

### Example 7

The Device of the Present Invention Mounted in Public Transport, Inside the Air Conditioning Duct, Using the Pre-Existing Structure and the Air Renewal System. Filters, Ionizers, UVC Light, Ozonizer, and Motion Sensors were Placed. A PLC that Commands the Entire Process was Installed.

Analysis microorganisms removal in buses.

### Objective

To determine the microbial reduction in controls (aerobic mesophiles and fungi and yeasts) due to their exposure to the air stream that circulates in a double-decker passenger bus owned by Chevallier Company, wherein said air stream has been exposed to filtration, UVC light and an ionizer.

### Development

In this test, two double-decker buses with identical volume characteristics and identical central air conditioning systems were used.

In one of them, the device of the present invention with HEPA filter, activated carbon filter, ionizer equipment and UVC lamp was installed. The other unit, used as a control, was not modified.

Additionally, some employees of the company got in the bus equipped with the device of the present invention (occupying approximately 50% of the bus capacity), in order to simulate travel conditions, in which the air flow would perform 20 cycles, in order to comply with the current regulations.

Sampling of different plates (3M™ Petrifilm™ Rapid Yeast and Mold Count Plates & Petrifilm™ Aerobic Count Plates) was performed with 1/10 dissolution. The visualization of a growth point on the plate; equals 10 CFU (CFU=Colony Forming Units). This unit is how microbial growth is expressed.

The standard microbiological air criteria are:

| | | |
|---|---|---|
| ENVIRONMENTAL | MESOPHILES: <500 | MOLD AND |
| SPECIFICATION *: | CFU | YEAST: <100 CFU |

* These specifications are based on reference publications provided by 3M ™ for indoor air quality and may be taken as valid as limits for outdoors.

Said plates were exposed in different areas of the units:
Passenger seats in the lower area. (APZB)
Passenger seats in the upper front area. (APZSD)
Passenger seats in the upper rear area. (APZST)

Table VI: results obtained in the laboratory, after incubation at controlled temperature.

| | | | |
|---|---|---|---|
| Device | Absent | Operating | % Reduction |

Mesophiles CFU

| | | | |
|---|---|---|---|
| APZB | 60 | 10 | 83.3% |
| APZSD | 70 | <10 | >85.7% |
| APZST | 60 | 20 | 66.7% |

Mold and yeasts CFU

| | | | |
|---|---|---|---|
| APZB | 70 | <10 | >85.7% |
| APZSD | 60 | <10 | >83.3% |
| APZST | 40 | 10 | 75% |

Table VI compares the microorganisms reduction (aerobic mesophiles and mold and yeasts), with and without the use of UVC (lamps).

Characteristics of bus air conditioning system:

Hourly air renewal on both floors 83 cycles/h.

In this way, the resulting time of exposure of the plates with and without UVC equipment was set at 20 min, to guarantee a minimum of 20 cycles or air renewals, complying the current requirements.

### Conclusions

According to the results presented, it is clear that even the most demanding condition, a bus with passengers obtains very significant reduction values.

The effect of the operation of the device of the present invention over microorganisms such as Aerobic Mesophiles and mold and yeasts, show the effectiveness of the device installed in the central air conditioning system, and such analysis can be extended to viral load.

Finally, the device installed as detailed in the example and used in these conditions would be a controlling element for a dynamic system such as the bus, complying with the air specifications set for this type environments.

### CITATIONS

Tang, Fan; Xie, Chu; Zhang, Xianji; Ceng, Xianying. (2019) Novel indoor air purifier with intelligent induction and control. CN105737262.
Maletich, Peter; Applegate, Shawn Michael; Rydholm, Mitch; Barnaby, Jr; Matlin, Tai Hoon K. (2018) Air purifier with intelligent sensors and airflow. WO2018/106809.
Wang, Jun. (2014). Novel negative ion air purifier. CN203785115.
Yao, Tieming. (2016). On-vehicle air purifier device. CN205615319.

1. A dual device for environments disinfection and sterilization of air and surfaces, comprising:
an external casing with mirrored inner surfaces;
a top intake of polluted air;
a bottom outlet of purified air;
a blower;
at least one HEPA filter;
at least one activated carbon filter;
at least one UV lamp;
an ionizer;
an ozonizer and
a buzzer.

2. The device of claim 1 wherein said top intake of polluted air is located in the upper section of said casing and said bottom outlet of purified air is located in the lower section of said casing.

3. The device of claim 1 including a photocatalytic air filter.

4. The device of claim 1 wherein it operates at a height of at least two meters attached to a wall, aspiring the air through its upper part and driving the purified air downwards, in such a way that in its sterilization mode of operation the ozone reaches all the surfaces of furniture and objects that are below two meters in height, achieving the sterilization of the surfaces.

5. The device of claim 1 further comprising a remote-controlled automatic control system; a presence sensor that detects the absence of living beings in the room where it is installed, enabling the operation of said ozonizer to comply with the sterilization mode of operation, where said sterilization mode also activates said buzzer that generates a light signal and a buzz that makes it impossible to stay in the room to be sterilized.

6. The device of claim 1 wherein said UV lamps, said activated carbon filter, said ionizer and said ozonizer are installed on aluminum profiles inside said casing constituted by the preexisting ducts of the air conditioning system of a vehicle, wherein said blower is the impeller of said air conditioning system.

7. The device of claim 6 wherein said activated carbon filter, said HEPA filter and said photocatalytic air filter are manufactured to fit in said ducts and the circulating air passes through them.

8. The device of claim 6 wherein a touch screen, the presence sensor, and all the electronic components necessary for its operation are installed according to the needs and possibilities in each vehicle.

9. The device of claim 1 wherein said activated carbon filter comprises granular, fiber or powdered activated carbon.

10. The device of claim 1 wherein said HEPA filters grades are selected from the group comprised by H11, H13 and H14.

11. The device of claim 1 wherein said ionizer is a plasma ionizer.

12. The device of claim 1 wherein the photocatalytic filter comprises a mesh structure coated by TiO₂ which is illuminated by UV lamps.

13. The device of claim 1 wherein said casing with mirrored inner surfaces comprises polished metal at least on its inner surface.

14. The device of claim 1 wherein said UV lamps are selected from the group comprised by: UVA/UVC lamps and UV-C FAR lamps.

15. The device of claim 14 wherein said UV lamps are selected from the group comprised by: LED technology lamps and mercury vapor lamps.

16. A method of purification, cleaning and disinfection of air and surfaces for indoors that uses the device of claim 1 wherein said device is preferably installed at a distance of at least 2 m and less than 5 m from the ground and, comprising the following steps:
a. aspiring the air from the room to purify through the top intake;
b. forcing the air aspired in step “a” to pass through the activated carbon filter, through the HEPA filter, through a UV lamp, through the ionizer and
c. expelling the purified and disinfected air through a bottom outlet.

17. A method of purification, cleaning and disinfection of air and surfaces for indoors that uses the device of claim 1 wherein said device is preferably installed at a distance of at least 2 m and less than 5 m from the ground and, comprising the following steps:
a. aspiring the air from the room to purify through the top intake;
b. forcing the air aspired in step “a” to pass through the activated carbon filter, through the HEPA filter, through a UV lamp, through the ionizer, through the ozonizer and
c. expelling the purified and disinfected air through a bottom outlet.

18. The method of claim 17, wherein it is a method of sterilizing the air of the room in which it is installed, which comprises the automatic activation of said ozonizer when its remote-controlled automatic control system detects the absence of living beings in the room.

19. The device of claim 1 wherein said blower comprises two centrifugal turbines commanded by a single electric motor located between said turbines that operates at 2800 rpm and generates an air flow of at least 2000 m³/h.

20. The device of claim 1 characterized in that said centrifugal turbines have a design with reduced air intake ducts.

21. The device of claim 1 wherein it comprises two modes of operation: standard and sterilization; the ozonizer only works in sterilization mode.
